(19) United States
(12) Patent Application Publication
(10) Pub. No.: US 2004/0235807 A1
(43) Pub. Date: Nov. 25, 2004

(54) Formulations including a topical decongestant and a topical corticosteroid suitable for nasal administration and method for treating obstructive sleep apnea

(76) Inventors: Karl P. Weinrich, Bridgewater, MA (US); Leonard W. Kaplan, Brookline, MA (US)

Correspondence Address: REED & EBERLE LLP, 800 MENLO AVENUE, SUITE 210, MENLO PARK, CA, 94025, US

(21) Appl. No.: 10/443,672

(22) Filed: May 21, 2003

(51) Int. Cl. A61K 31/573; A61K 31/137

(52) U.S. Cl. 514/171; 514/649

(57) ABSTRACT

A pharmaceutical formulation is provided for nasal drug administration of a topically administrable decongestant, a topically administrable corticosteroid and a pharmaceutically acceptable carrier, and may optionally include further carriers, therapeutic extenders, and the like. Such formulations may also optionally further include a therapeutically active member selected from the group consisting of a topical antibiotic, a topical antihistamine (preferably a non-sedating antihistamine), a leukotriene D₄ antagonist, a 5-lipoxygenase inhibitor, and a FLAP antagonist, or a pharmaceutically acceptable salt thereof. In addition, methods for using the formulation to treat decongestant/corticosteroids-responsive conditions, diseases or disorders such as chronic obstructive nasal congestion and/or obstructive sleep apnea conditions, are provided, as are drug delivery devices and dosage forms for housing and/or dispensing the formulations.

### TECHNICAL FIELD

This invention relates generally to pharmaceutical formulations, and more particularly relates to pharmaceutical formulations comprising at least one topical corticosteroid and at least one topical nasal decongestant (e.g., sympathomimetic amine), and one or more optional members selected from the group consisting of carriers, extenders and excipients. In addition, the invention relates to methods of using the described formulations and drug delivery devices containing the described formulations in the treatment of chronic nasal congestion and/or in the treatment of obstructive sleep apnea.

### BACKGROUND ART

In a normal person, the nasal turbinates, small, shelf-like, structures composed of thin bone and covered by mucous membranes (mucosal), protrude into the nasal airway and help to warm, humidify and cleanse air as it is inhaled and before it reaches the lungs. Since the nasal airway is the normal breathing route during sleep, chronic enlargement (hypertrophy) of the nasal turbinates (caused by conditions such as chronic nasal congestion) can contribute to headaches and cause sleep disorders such as snoring and obstructive sleep apnea. Such chronic turbinates hypertrophy and nasal obstruction are commonly associated with chronic rhinitis (the inflammation of the mucosal lining of the nose). When the mucosal tissue becomes inflamed, the blood vessels inside the membrane swell and expand, causing the turbinates to become enlarged and obstruct the flow of air through the nose. According to several large populations surveys, approximately 20% of the population, or more than 50 million Americans, suffer from some type of chronic rhinitis.

Medications designed to treat the stuffy nose, sinus complaints and the common cold make up the largest segment of the over-the-counter drug market for the U.S. pharmaceutical industry, accounting for nearly $3.5 billion in sales. First-line medical treatment for the chronic stuffy nose and chronically enlarged turbinates associated with rhinitis mainly consists of a variety of antihistamines, decongestants, and topical and systemic corticosteroids. Surgery to reduce the size of the nasal turbinates with a concurrent reduction of symptoms is sometimes utilized, but this does not entirely cure such conditions in many patients. Post surgery continued swelling of the mucosal lining throughout the nasal passages (and deep in the sinuses) continues to aggravate the patients' condition with a return of symptoms and conditions. Accordingly, surgery often does not cure or give adequate chronic relief for the underlying condition.

Topical decongestants (as nasal sprays) are used to treat a chronic stuffy nose, but such use is not without serious drawbacks. Such topical decongestants act by constricting the blood vessels in swollen mucous membranes, forcing blood out so that the membranes shrink and air passages open. Typical commercial nasal sprays contain sympathomimetic compounds (decongestants), such as 0.05% oxymetazoline in Afrin® and the like. Common over-the-counter remedies contain phenylephrine hydrochloride, oxymetazoline hydrochloride, xylometazoline hydrochloride, and the like. These topical decongestants are generally considered to be harmful when used over long periods of time because they cause damage to nasal mucosal ciliary function, and they cause rebound mucosal thickening leading to nasal congestion. The manufacturers of certain over-the-counter remedies often warn that their products should not be used for more than three days. The use or abuse of these drugs can result in prolonged nasal obstruction (“rebound” can be worse than initial symptoms), where addictive use behavior can occur to avoid such uncomfortable rebound nasal congestion.

Several corticosteroid therapies, mostly in the form of a nasal spray or inhaler, have been developed to treat chronic nasal obstruction. Intranasal corticosteroid sprays are available only by prescription and they can be very effective, however, they are associated with side effects such as bleeding, drying and crusting. Patients must take care not to overuse corticosteroid preparations. Although the drugs are applied topically, some systemic absorption of the agent occurs, which can disrupt the body's steroid balance. Steroids can also be injected directly into the turbinates, however, their effectiveness usually lasts only three to six weeks. The anti-inflammatory effect of steroid sprays may produce a beneficial reaction in the nasal and sinus mucosa. However, such sprays are generally effective only in reducing inflammation, and only have inconsequential decongestant or physiological mucosal effects in mobilizing secretions or stimulating cells to evacuate secretions.

As mentioned above, chronic enlargement (hypertrophy) of the nasal turbinates (caused by conditions such as chronic nasal congestion) can cause sleep disorders such as snoring and obstructive sleep apnea. Obstructive sleep apnea (also referred to herein as “OSA”) is a breathing disorder that occurs primarily during sleep with consequences that may persist throughout the waking hours in the form of sleepiness, thereby manifesting itself into substantial economic loss (e.g., thousands of lost man-hours) or employment safety factors (e.g., employee non-attentiveness during operation of heavy-machinery). Such sleep-related breathing disorders are characterized by repetitive reduction in breathing (hypopnea), periodic cessation of breathing (apnea), or a continuous or sustained reduction in ventilation. OSA is a significant public health burden. Such OSA disorder and related symptoms affects all races, ages and socioeconomic and ethnic groups. There is no known pharmacological treatment for OSA on the market that is generally successful.

In general, sleep apnea is defined as an intermittent cessation of airflow at the nose and mouth during sleep. By convention, apneas of at least 10 seconds in duration have been considered important, but in most individuals the apneas are 20-30 seconds in duration and may be as long as 2-3 minutes. While there is some uncertainty as to the minimum number of apneas that should be considered clinically important, by the time most individuals come to attention of the medical community they have at least 10 to 15 events per hour of sleep.

Sleep apneas have been classified into three types: central, obstructive, and mixed. In central sleep apnea the neural drive to all respiratory muscles is transiently abolished. In obstructive sleep apneas, airflow ceases despite continuing respiratory drive because of occlusion of the oropharyngeal airway. Mixed apneas, which consist of a central apnea followed by an obstructive component, are a variant of obstructive sleep apnea. The most common type of apnea is OSA.

OSA syndrome has been identified in as many as 24% of working adult men and 9% of similar women, with peak prevalence in the sixth decade. Habitual heavy snoring, which is an almost invariant feature of OSA, has been described in up to 24% of middle aged men, and 14% of similarly aged women, with even greater prevalence in older subjects.

OSA syndrome's definitive event is the occlusion of the upper airway, frequently at the level of the oropharynx. The resultant apnea generally leads to a progressive-type asphyxia until the individual is briefly aroused from the sleeping state, thereby restoring airway patency and thus restoring airflow. An important factor that leads to the collapse of the upper airway in OSA is the generation of a critical sub-atmospheric pressure during the act of inspiration that exceeds the ability of the airway dilator and abductor muscles to maintain airway stability. Sleep plays a crucial role by reducing the activity of the muscles of the upper airways including the dilator and abductor muscles.

In individuals with OSA, in addition to chronic congestive nasal obstruction, the airway can be compromised structurally and be predisposed to occlusion. Obesity also frequently contributes to a reduction in size seen in the upper airways. The act of snoring, which is actually a high-frequency vibration of the palatal and pharyngeal soft tissues that results from the decrease in the size of the upper airway lumen, usually aggravates the narrowing via the production of edema in the soft tissues.

The recurrent episodes of nocturnal asphyxia and of arousal from sleep that characterize OSA lead to a series of secondary physiologic events, which in turn give rise to the clinical complications of the syndrome. The most common manifestations are neuro-psychiatric and behavioral disturbances that are thought to arise from the fragmentation of sleep and loss of slow-wave sleep induced by the recurrent arousal responses. Nocturnal cerebral hypoxia also may play an important role. The most pervasive manifestation is excessive daytime sleepiness. OSA is now recognized as a leading cause of daytime sleepiness and has been implicated as an important risk factor for such problems as motor vehicle accidents. Other related symptoms include intellectual impairment, memory loss, personality disturbances, and impotence.

The other major manifestations are cardio-respiratory in nature and are thought to arise from the recurrent episodes of nocturnal asphyxia. Most individuals demonstrate a cyclical slowing of the heart during the apneas to 30 to 50 beats per minute, followed by tachycardia of 90 to 120 beats per minute during the ventilatory phase. A small number of individuals develop severe bradycardia with asystoles of 8 to 12 seconds in duration or dangerous tachyarrhythmias, including unsustained ventricular tachycardia. OSA also aggravates left ventricular failure in patients with underlying heart disease. This complication is most likely due to the combined effects of increased left ventricular afterload during each obstructive event, secondary to increased negative intrathoracic pressure, recurrent nocturnal hypoxemia, and chronically elevated sympathoadrenal activity.

Currently, the most common and most effective treatment for adults with sleep apnea and other sleep-related breathing disorders are mechanical forms of therapy that deliver continuous positive airway pressure (also referred to herein as “CPAP”). Under CPAP treatment, an individual wears a tight-fitting plastic mask over the nose when sleeping. The mask is attached to a compressor, which forces air into the nose creating a positive pressure within the patient's airways. The principle of the method is that pressurizing the airways provides a mechanical “splinting” action, which prevents airway collapse and therefore prevents OSA. Although an effective therapeutic response is observed in most patients who undergo CPAP treatment, many patients cannot tolerate the apparatus or pressure and refuse treatment. Moreover, recent covert monitoring studies clearly demonstrate that long-term compliance with CPAP treatment is very poor.

A variety of upper airway and craniofacial surgical procedures have been attempted for treatment of OSA. Adenotonsillectomy appears to be an effective cure for OSA in many children, but upper airway surgery is rarely curative in adult patients with OSA. Surgical “success” is generally taken to be a 50% reduction in apnea incidence and there are no useful screening methods to identify the individuals that would benefit from the surgery versus those who would not derive a benefit.

Pharmacological treatments of several types have been attempted in patients with sleep apnea but, thus far, none have proven to be generally useful. A recent systematic review of these attempts is provided by Hudgel (1995) J. Lab. Clin. Med. 126:13-18. A number of compounds have been tested because of their expected respiratory stimulant properties. These include (1) acetazolamide, a carbonic anhydrase inhibitor that produced variable improvement in individuals with primary central apneas but caused an increase in obstructive apneas, (2) medroxyprogesterone, a progestin that has demonstrated no consistent benefit in OSA, and (3) theophylline, a compound usually used for the treatment of asthma, which may benefit patients with central apnea but appears to be of no use in adult patients with obstructive apnea.

Other attempted pharmacological treatment includes the administration of adenosine, adenosine analogs and adenosine reuptake inhibitors (U.S. Pat. No. 5,075,290 to Findley, et al.). Specifically, adenosine, which is a ubiquitous compound within the body and which levels are elevated in individuals with OSA, has been shown to stimulate respiration and is somewhat effective in reducing apnea in an animal model of sleep apnea.

Other possible pharmacological treatment options for OSA include agents that stimulate the brain activity or agents that are opioid antagonists. Specifically, since increased cerebral spinal fluid opioid activity has been identified in OSA, it is a logical conclusion that central stimulants or opioid antagonists would be a helpful treatment of OSA. In reality, doxapram, which stimulates the central nervous system and carotid body chemoreceptors, was found to decrease the length of apneas but did not alter the average arterial oxygen saturation in individuals with OSA. The opioid antagonist naloxone, which is known to stimulate ventilation, was only slightly helpful in individuals with OSA.

Because OSA syndrome is strongly correlated with the occurrence of hypertension, agents such as angiotensin-converting enzyme (ACE) inhibitors may be of benefit in treating OSA individuals with hypertension but do not appear to be a viable treatment for OSA itself.

Finally, several agents that act on neurotransmitters and neurotransmitter systems involved in respiration have been tested in individuals with OSA. Most of these compounds have been developed as anti-depressant medications that work by increasing the activity of monoamine neurotransmitters, including norepinephrine, dopamine, and serotonin. Protriptyline, a tricyclic anti-depressant, has been tested in several small trials with variable results and frequent and significant side effects. As serotonin may promote sleep and stimulate respiration, tryptophan, a serotonin precursor and selective serotonin reuptake inhibitors have been tested in individuals with OSA. While a patent has been issued for the use of the serotonin reuptake inhibitor, fluoxetine (U.S. Pat. No. 5,356,934 to Robertson, et al.), initial evidence suggests that these compounds may yield measurable benefits in only approximately 50% of individuals with OSA. Therefore, in view of the fact that the only viable treatment for individuals suffering from sleep-related breathing disorders is a mechanical form of therapy (CPAP) for which patient compliance is low, and that hopes for pharmacological treatments have yet to come to fruition, there remains a need for simple pharmacologically-based treatments that would offer benefits to a broad base of individuals suffering from a range of sleep-related breathing disorders. There also remains a need for a viable treatment of sleep-related breathing disorders that would lend itself to a high rate of patient compliance.

In view of the above, current drugs only provide temporary symptomatic improvement and symptom reduction for chronic congestive nasal obstruction that can lead to OSA, but due to significant side effects must be rotated to avoid addition and subsequent rebound of symptoms. Surgery to reduce the size of the nasal turbinates is also sometimes utilized (as described above) to reduce symptoms in an attempt to treat sleep apnea, but this does not entirely cure such conditions in many patients. Post surgery continued swelling of the mucosal lining throughout the nasal passages and in the sinuses continues to aggravate the patients' condition with a return of symptoms, and thus, do not cure or give chronic relief for the underlying condition.

There is, accordingly, a need in the art to provide a composition for the effective, safe, and long-term treatment of OSA and/or chronic nasal obstruction that may be related to OSA, which reduces significant side effects and also reduces withdrawal rebound nasal congestion.

### SUMMARY OF THE INVENTION

One primary aspect of the invention is a method for treating a patient suffering from a chronic condition, disease or disorder that is responsive to treatment with a decongestant/corticosteroid combination, comprising nasally administering to the patient a pharmaceutical formulation for nasal drug administration, wherein the formulation comprises: a therapeutically effective amount of a topically administrable decongestant; a therapeutically effective amount of a topically administrable corticosteroid; and a pharmaceutically acceptable carrier that is suitable for nasal drug administration.

Another aspect of the invention is a method for the treatment of a patient suffering from chronic nasal congestive obstruction or obstructive sleep apnea (OSA) with a topical decongestant/corticosteroid combination, comprising nasally administering to the patient a pharmaceutical formulation for topical drug administration as described above. This method may provide for the long-term treatment of such conditions.

Yet another aspect of the invention is a long-term treatment method for treating chronic nasal congestion and/or treating of OSA comprising daily administration of nasal pharmaceutical formulations that are adapted for such long-term administration (as described below), as well as drug delivery devices containing such formulations and means for daily administration in such methods.

Other aspects of the invention involve the methods described above, that provide for the administration of a pharmaceutical composition that comprises at least one topically administrable nasal sympathomimetic amine decongestant, which is a member selected from the group consisting of: oxymetazoline, xylometazoline, naphazoline, phenylephrine and pharmaceutically acceptable salts thereof.

Still another aspect of the invention is a method that includes the administration of a composition, as described above, and may optionally further include a therapeutically active agent selected from the group consisting of a topical antibiotic, a topical antihistamine (preferably a non-sedating antihistamine), a leukotriene D₄ antagonist, a 5-lipoxygenase inhibitor, and a 5-lipoxygenase-activating protein (FLAP) antagonist, or a pharmaceutically acceptable salt thereof.

Another aspect of the invention is a pharmaceutical composition for nasal drug administration comprising: a therapeutically effective amount of a topically administrable decongestant; a therapeutically effective amount of a topically administrable corticosteroid; and a pharmaceutically acceptable carrier that is suitable for nasal drug administration.

Still another aspect of the invention is a nasal or sinus drug delivery device, comprising: a topically administrable pharmaceutical formulation as described above, and a means for housing and dispensing unit dosages of the formulation into a patient's nasal passages and/or neighboring sinuses. A preferred drug delivery device comprises an aqueous extender solvent system, whereby each dose delivered has the ability to maintain a therapeutic effect for a period of more than 6 hours.

Still another aspect of the invention is a dosage form containing a topically administrable nasal pharmaceutical formulation as described above, and optionally a unit dose delivery system. The system may include a means for housing and dispensing metered unit dosages of the formulation into a patient's nasal passages and/or into the neighboring sinuses.

Additional aspects, advantages and features of the invention will be set forth in part in the description that follows, and in part will become apparent to those skilled in the art upon examination of the following, or may be learned by practice of the invention.

### DETAILED DESCRIPTION OF THE INVENTION

This invention is based, in part, upon the unexpected discovery that combination treatment of a patient with a nasally administrable decongestant and corticosteroid provides a treatment regimen wherein the adverse effects attributable to each member of the combination are essentially eliminated by the other member of the combination.

In one embodiment, the invention provides a method for treating a patient suffering from a condition, disease or disorder that is responsive to treatment with a decongestant/corticosteroid combination, comprising nasally administering to the patient a pharmaceutical formulation for nasal drug administration, wherein the formulation comprises: (i) a therapeutically effective amount of a topically administrable decongestant; (ii) a therapeutically effective amount of a topically administrable corticosteroid; and (iii) a pharmaceutically acceptable carrier that is suitable for nasal drug administration. Any topically administrable decongestant may be utilized in the invention, including pharmacologically acceptable salts and esters thereof, as well as combinations of topical decongestants suitable to treat nasal or sinus congestion, may be included in the formulation. It is preferred, however, that the decongestant present be a sympathomimetic amine. Topical decongestants in this class include, for example, oxymetazoline, xylometazoline, naphazoline, phenylephrine and pharmnaceutically acceptable salts thereof.

Although any salt, ester or derivative of a corticosteroid that will yield a therapeutically effective topical metabolite may serve as the topically administrable corticosteroid in the above method, it is particularly preferred that an ester form, e.g., acetate form, thiophene ester form or furoate form is present in the formulation. Topical corticosteroids in this class include, for example, beclomethasone, budesonide, flunisolide, fluticasone, flunidolone, mometasone, triamcinolone, dexamethasone, and pharmaceutically acceptable salts, esters, or derivatives thereof.

In another embodiment, the invention provides a pharmaceutical composition for nasal drug administration, comprising: a therapeutically effective amount of a topically administrable decongestant; a therapeutically effective amount of a topically administrable corticosteroid; and a pharmaceutically acceptable carrier that is suitable for nasal drug administration.

The topically administrable pharmaceutical formulations provided by the present invention are particularly well suited to the long-term treatment of patients suffering from chronic obstructive nasal congestion and/or obstructive sleep apnea, as well as for the optional treatment of other conditions that may simultaneously afflict a patient, such as, for example, nasal or sinus allergic conditions, inflammation, or infections. However, the formulations can be tailored to be effective in the treatment of patients suffering from both acute and chronic episodes of these maladies.

In yet another embodiment, a drug delivery device is provided comprising a pharmaceutical formulation as described herein and a means for housing and dispensing unit dosages of the formulation. The drug delivery device may be any device that is effective in delivering the formulation to the nasal system and the nasal/cranial sinus system. Thus, for example, the drug delivery device may be a dry powder inhaler, a metered-dose inhaler (MDI), a nebulizer or a pump spray bottle. A dry powder inhaler is one particularly preferred device for delivering the formulations of the invention. Another preferred device is an aqueous based pump spray capable of delivering metered dosages of the formulations of the invention.

### I. Definitions and Nomenclature

Unless otherwise indicated, the invention is not limited to specific synthetic methods, analogs, substituents, pharmaceutical formulations, formulation components, modes of administration, delivery systems, or the like, as such may vary. It is also to be understood that the terminology used herein is for the purpose of describing particular embodiments only, and is not intended to be limiting.

It must be noted that, as used in this specification and the appended claims, the singular forms “a,” “an” and “the” include plural referents unless the context clearly dictates otherwise. Non-limiting examples illustrating plural inclusion in the present specification are: references to “a decongestant” which term includes a combination of two or more decongestants, and when referring to a decongestant (such as oxymetazoline) without specifying a salt, ester, therapeutically active metabolite, or therapeutically active derivative also encompasses such structures and combinations thereof; references to “a corticosteroid” which term includes combinations of two or more corticosteroids and when referring to a corticosteroid (such as mometasone) without specifying a salt, ester, therapeutically active metabolite, or therapeutically active derivative includes such structures and combinations thereof; references to “a pharmaceutically acceptable carrier” includes liquid, solid or gaseous carriers and includes combinations of two or more pharmaceutically acceptable carriers; and the like. It will be understood by one skilled in this technology that a carrier may also optionally perform the function of a therapeutic “extender”, whereby the carrier may provide the therapeutic effect of extending the release time for an active agent in the formulation, or otherwise prolong the time for an active agent's therapeutic effect.

If utilized in the specification and claims directed to the present invention, the following terminology will be used in accordance with the definitions set forth below.

The terms “active agent,” “drug” and “pharmacologically active agent” are used interchangeably herein to refer to a chemical material, composition or compound which, when administered to an organism (human or animal), induces a desired pharmacologic effect or activity. Such terms (and corresponding specified molecular entity examples) encompass not only a specified molecular entity, particular chemical structure, or root molecular structure, but also encompass pharmaceutically acceptable, pharmacologically active analogs, including, but not limited to, salts, esters, amides, prodrugs, conjugates, active metabolites, and other such derivatives, analogs, and related compounds which can be formulated to induce the desired pharmacologic activity (e.g., an active agent, such as a decongestant agent, might be recited in the specification or claims merely as “oxymetazoline” while this term encompasses all the many active salts, derivatives and the like that are related to, or derived from, oxymetazoline and have decongestant activity).

By “nasal administration” of a “topical drug” or “topically active drug” it is meant that the drug is applied to, or into, bodily tissues by some means or manner through the nares (nostrils) of a patient, and thereby has a resulting therapeutic effect upon one or more of: tissues in the nasal passages, tissues in the upper respiratory sinuses that are open to (or accessible from) the nasal passages, and other upper respiratory tissues, or may have a resulting optional systemic therapeutic effect, wherein such effect(s) is/are primarily a result of topical absorption or infusion into tissues to which the drug is administered, or is/are a result of topically protecting or shielding such tissues from contact with environmental (or therapeutic) conditions or agents. Such administration (of an active ingredient to any part, tissue or organ that is directly or indirectly involved with the upper respiratory tract that when inflamed or swollen can lead to chronic obstructive nasal congestion and/or obstructive sleep apnea) is accomplished by administering an active ingredient through one or more nasal openings. The above term is intended to contemplate the upper airway passages and include, for example, the back of the mouth or throat, nose, pharynx, oropharynx, laryngopharynx, larynx, the inner ear passages, and the sinuses that open into or drain into such areas. Thus, the phrase “nasal administration” includes administering of the formulation described herein to any part, tissue or organ within a patient that is directly or indirectly involved with either the external and internal nasal passages, or involved with the nasal sinuses.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, “treating” a patient with a compound of the invention includes prevention of a particular disorder or adverse physiological event in a susceptible individual as well as treatment of a clinically symptomatic individual by inhibiting or causing regression of a disorder or disease. For example, treatment of OSA encompasses not only treatment that causes a regression or diminution of current symptoms, but also involves chemoprevention treatment in a patient susceptible to developing OSA (e.g., at a higher risk, as a result of genetic predisposition, environmental factors, or the like) and/or chemoprevention in post-surgery OSA patients at risk of OSA recurrence, as well as the treatment of an OSA patient by dual inhibiting or causing regression of a disorder or disease. Therefore, the terms “treating” and “treatment” as used herein may refer to a reduction in severity and/or frequency of symptoms, elimination of symptoms and/or underlying cause, prevention of the occurrence of symptoms and/or their underlying cause, and improvement or remediation of damage. For example, the present method of “treating” chronic nasal obstructive congestion and/or OSA, as the term “treating” is used herein, encompasses both prevention of these conditions in a predisposed individual and treatment of such conditions in a clinically symptomatic individual.

The terms “condition,” “disease” and “disorder” are used interchangeably herein as referring to a physiological state that can be prevented or treated by administration of a pharmaceutical formulation as described herein.

The term “patient” as in treatment of “a patient” refers to a mammalian individual afflicted with or prone to a condition, disease or disorder as specified herein, and includes both humans and animals.

By the terms “effective amount” and “therapeutically effective amount” of a compound of the invention is meant a nontoxic but sufficient amount of the drug or agent to provide the desired effect. The exact amount required will vary from subject to subject, depending on the age, weight, and general condition of the subject, the severity of the condition being treated, the judgment of the clinician, and the like. Thus, it is not possible to specify an exact “effective amount.” However, an appropriate “effective” amount in any individual case may be determined by one of ordinary skill in the art using routine experimentation.

The term “dosage form” denotes any form of a pharmaceutical composition that contains an amount of active agent sufficient to achieve a therapeutic effect with a single administration. When the formulation is a tablet or capsule, the dosage form is usually one such tablet or capsule. The frequency of administration that will provide the most effective results in an efficient manner without overdosing will vary with the characteristics of the particular active agent, including both its pharmacological characteristics and its physical characteristics, such as hydrophilicity.

The term “controlled release” refers to a drug-containing formulation or fraction thereof in which release of the drug is not immediate, i.e., with a “controlled release” formulation, administration does not result in immediate release of the drug into an absorption pool. The term is used interchangeably with “nonimmediate release” as defined in Remington: The Science and Practice of pharmacy, Twentieth Ed. (Easton, Pa.: Mack Publishing Company, 2000). In general, the term “controlled release” as used herein includes sustained release and delayed release formulations, and both types of release are optimally intended to prolong the time of a therapeutic effect for an active ingredient.

The term “sustained release” (synonymous with “extended release”) is used in its conventional sense to refer to a drug formulation that provides for gradual release of a drug over an extended period of time, and that preferably, although not necessarily, results in substantially constant blood levels of a drug over an extended time period.

By “pharmaceutically acceptable” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration. “Pharmacologically active” (or simply “active”) as in a “pharmacologically active” derivative or analog, refers to a derivative or analog having the same type of pharmacological activity as the parent compound and approximately equivalent in degree.

“Optional” or “optionally” means that the subsequently described event or circumstance may or may not occur, and that the description includes instances where said event or circumstance occurs and instances where it does not. For example, reference to an “optional pharmaceutically acceptable carrier” in a formulation indicates that such a carrier may or may not be present, and the description includes formulations wherein a carrier is present and formulations wherein a carrier is not present.

### II. The Pharmaceutical Formulations

The invention, as noted above, is in one embodiment a pharmaceutical formulation for nasal drug administration, comprising: a topically administrable decongestant and a topically administrable corticosteroid and a pharmacologically carrier, in either a dry powder form or in an aqueous vehicle form. Such an aqueous vehicle system and an optional polymeric carrier and therapeutic extender, as described in U.S. Pat. No. 6,316,483, to Hanswalter et al., may be utilized as the aqueous vehicle in the present invention (the '483 patent describes intra-nasal compositions comprising a topical decongestant, an antihistamine and a polymeric extender (a corticosteroid was neither described or indicated in that document)). Typical carriers (excipients), preservatives, and the like, as well as delivery systems, were also described in the '483 patent and are incorporated herein entirely by reference. Thus, the formulations described herein include at least two active agents, i.e., a topical decongestant and a topical corticosteroid and a pharmacologically acceptable carrier. In one embodiment of the present invention (when an optional antihistamine is incorporated) the compositions as described in the '483 patent may be utilized as an intermediate composition to which is added a therapeutically effective amount of a corticosteroid selected from the group consisting of beclomethasone, budesonide, pharmaceutically acceptable salts, esters, or derivatives and mixtures thereof.

The formulation of the present invention may also contain additional carriers (or excipients), or therapeutic extenders, provided that such additional ingredients do not have a deleterious effect on the intended patient or have a deleterious chemical or physical effect on any component in the formulation. Thus, for example, carriers such as preservatives, surface active agents, buffering agents, suspending agents, and the like can be combined with the formulation. The type and amount of any carrier will depend on the type of formulation and the device used for administration, as will be appreciated by one of ordinary skill in the art. Specific examples of each of these carriers are well known by those skilled in the art of pharmaceutical formulation.

### A. Active Agents

Any of the active agents in the formulation may be administered in the form of a pharmacologically acceptable salt, ester, amide, prodrug, active metabolite, derivative, or the like, or as a combination thereof. Salts, esters, amides, prodrugs, active metabolites, analogs, and other derivatives of the active agents may be prepared using standard procedures known to those skilled in the art of synthetic organic chemistry and described, for example, by J. March (1992), “Advanced Organic Chemistry: Reactions, Mechanisms and Structure,” New York: Wiley-Interscience 4.

For example, acid addition salts may be prepared from the free base (e.g., compounds having a neutral -NH₂ or cyclic amine group) using conventional means, involving reaction of the free base with a suitable acid. Typically, the base form of an active agent is dissolved in a polar organic solvent such as methanol or ethanol and the acid is added at a temperature of about 0° C. to about 100° C., preferably at ambient temperature. The resulting salt either precipitates or may be brought out of solution by addition of a less polar solvent. Suitable acids for preparing the acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like as well as inorganic acids, e.g., hydrochloric acid, hydrobromic acid, sulfuric acid, nitric acid, phosphoric acid, and the like. An acid addition salt may be reconverted into the free base by treatment with a suitable base. Conversely, basic addition salts of an active agent having an acid moiety (e.g., carboxylic acid group or hydroxyl group) are prepared in a similar manner using a pharmaceutically acceptable base. Suitable bases include both inorganic bases, e.g., sodium hydroxide, potassium hydroxide, ammonium hydroxide, calcium hydroxide, magnesium hydroxide, and the like, as well as organic bases such as trimethylamine, diethylmethylamine or the like. Preparation of esters involves functionalization of hydroxyl and/or carboxyl groups that may be present within the molecular structure of the drug. The esters are typically acyl-substituted derivatives of free alcohol groups, i.e., moieties which are derived from carboxylic acids of the formula RCOOH (or the corresponding acyl chloride) where R is alkyl, and preferably is lower, i.e., C₁ to C₆, alkyl. Esters can be reconverted to the free acids, if desired, by using conventional hydrogenolysis or hydrolysis procedures. Amides may be prepared from esters, using suitable amine reactants, or they may be prepared from an anhydride or an acid chloride by reaction with ammonia or a lower alkyl amine. Prodrugs, conjugates, and active metabolites may also be prepared using techniques known to those skilled in the art or described in the pertinent literature. Prodrugs and conjugates are typically prepared by covalent attachment of a moiety that results in a compound that is therapeutically inactive until modified by an individual's metabolic system. Other derivatives of the active agents may be prepared using standard techniques known to those skilled in the art of synthetic organic chemistry, or may be deduced by reference to the pertinent literature and texts.

Stereoisomers of the active agents are also included as part of the formulations described herein. A Stereoisomers is a compound having the same molecular weight, chemical composition, and constitution as another, but with the atoms arranged differently. That is, certain identical chemical moieties are at different orientations in space. This difference has the consequence of rotating the plane of polarized light. A pair of stereoisomers that are mirror images of each other are defined as enantiomers. Individual stereoisomers or enantiomers may have unique or beneficial properties that make that individual isomer particularly well suited for the present invention. Consequently, individual stereoisomers or enantiomers and mixtures thereof of the active agents are included as part of the invention. Thus, each active agent may be present in the formulation as (i) a 50/50 racemate, i.e., equal amounts of each enantiomer, (ii) an enantiomerically pure form, e.g., only the (S) or (R) form, or (ii) a racemic mixture of non-equal amounts of each enantiomer, e.g., nonequal amounts of the (S)/(R) enantiomers.

The various hydrates of the active agents are also included in the formulations of the invention. As is known, one or more water molecules may associate with a particular compound based on, for example, the availability of hydrogen bonding. Methods of producing hydrated species are known and include, for example, placing the active agent in a humid environment. In addition, methods of removing one or more water molecules are known and include, by way of example, exposing the active agent to dry heat.

1. Decongestants

The invention is not limited with respect to the particular nasal decongestants listed herein. Any topically administrable decongestant may be incorporated into the formulations. It is preferred, however, that the decongestant be a sympathomimetics amine agent selected from the group consisting of oxymetazoline, xylometazoline, naphazoline, phenylephrine and pharmaceutically acceptable salts thereof. Furthermore, the formulation is not limited to one decongestant as combinations of decongestants may also be present. The decongestant may be present in the formulation as a salt, ester, amide, metabolite, prodrug, or other derivative, or may be functionalized in various ways as will be appreciated by those skilled in the art.

Typically, compositions according to the present invention will contain a unit dose of from 1 to 100 micrograms of topical decongestant. The weight percentage of decongestant in the formulation is typically from 0.001% to 0.2% by weight, more preferably from 0.01% to 0.1% by weight. For example, Phenylephrine is typically marketed in composition concentrations of 0.25% and 0.5%, while oxymetazoline and xylometazoline are typically marketed in composition concentrations of 0.05% or 0.025%. Therefore the preferred weight range is different for different activity levels, but such preferred ranges are easily determined by activity comparisons with the above compositions. Also, naphazoline is used for ophthalmic applications at 0.1%, but the optimum level for administering intranasally might be different.

2. Corticosteroids

Any topically administrable corticosteroid may be utilized in the invention. Particularly preferred corticosteroids include beclomethasone, budesonide, flunisolide, fluticasone, flunidolone, mometasone, triamcinolone, dexamethasone, and mixtures thereof. Such may be in the form of pharmaceutically acceptable salts, esters, prodrugs, metabolites, or derivatives thereof that may be included in the present formulation. Pharmacologically acceptable esters of such compounds are preferred, with those derived from C₂ to C₆ carboxylic acids are particularly preferred. More specifically, the esters may be derived from corresponding carboxylic acids having two to six carbon atoms that are branched, unbranched or cyclic, saturated or unsaturated, aromatic or nonaromatic, and heteroatom substituted or unsubstituted. Typically, compositions according to the present invention will contain a unit dose of from 10 to 100 micrograms of topical corticosteroid. While lower doses should be effective in the current combination composition, the unit dose amount of a corticosteroid in the combination composition can typically be up to the same unit dose that is typically observed when the particular corticosteroid is administered as a solo therapeutic. For example, fluticasone is labeled to deliver 0.05 mg per spray, budesonide is labeled to deliver either 0.032 mg or 0.064 mg per spray, mometasone furoate monohydrate is labeled to deliver 0.05mg base equivalent per spray, while cromolyn sodium is labeled to delivers 5.2 mg per spray. Accordingly, preferred amounts of a particular corticosteroid unit doses will be different, depending upon activity, either more or less.

3. Additional Active Agents

The formulations of the present invention may contain other active and inactive agents that enhance, extend or otherwise prolong the therapeutic effect or the deliver of the combination of decongestant/corticosteroids to the patient according to the present invention.

In one embodiment, the additional pharmaceutically active agent is selected from an antibiotic, an anticholinergic, an antihistamine, a leukotriene D₄ antagonist, a 5-lipoxygenase inhibitor, a FLAP antagonist and combinations thereof.

For example, if the pharmaceutical compositions according to the present invention are to be indicated for concurrent treatment of an allergic disorder, additional optional therapeutic agents (in addition to or in place of the antihistamines, as described above) leukotriene inhibitor, lipoxygenase inhibitors, or 5-lipoxygenase-activating protein (FLAP) inhibitors may be added. Such compositions may contain one or more additional agents selected from: (i) a leukotriene D₄ antagonist which influences leukotriene action, such as montelukast, zafirlukast or pranlukast, (ii) a 5-lipoxygenase inhibitor, such as zileuton, piriprost or AWD 23-115, or (iii) a FLAP (5-lipoxygenase activating protein) antagonist, such as MK-591, MK-886, Bay 1005, which are suitable for easy topical administration, for example in the form of sprays or powders. The typical dose for a topical leukotriene D₄ antagonist is between 0.1% to 5% by weight, preferably 0.5 to 2% by weight, and more preferably from 0.8 to 1% by weight; the typical dose for a 5-lipoxygenase inhibitor is between 0.05 to 5% by weight, preferably, 0.25 to 2% by weight, and more preferably from 0.5 to 1% by weight; and the typical dose for a FLAP inhibitor is between 0.05 to 5% by weight, preferably, 0.25 to 2% by weight, and more preferably from 0.5 to 1% by weight.

An antihistamine may optionally be present in the formulation according to the invention in 0.001% to 2.0% by weight (with a range of from 0.1 to 0.5 by weight being preferred, but depending upon the activity of the particular antihistamine). Particularly preferred antihistamine are pheniramine, pyrilamine, barbinoxamine, chlorpheniramine, tripolidine, phenyltoloxamine, carbinoxamine, promethazine, brompheniramine, bromodiphenhydramine, dexbrompheniramine, loratadine, fexofenadine, cetirizine, terfenadine, astemizole, hydroxyzine, and the like, or a pharmaceutically acceptable, salt, ester, prodrug, metabolite, or derivative thereof.

B. Other Ingredients

A therapeutic nasal spray extender such as various gums and polymers can be evaluated to determine their suitability as bioadhesives to extend the nasal muco-cilia clearance time of nasal spray formulations. Desired properties of a bioadhesive include solubility, clarity and compatibility in a conventional nasal spray formulation. In addition, the nasal spray composition containing the bioadhesive material can be evaluated to determine the concentration effect on spray pattern and resultant mist properties. Examples of extenders include methylcellulose, hydroxypropylmethylcellulose, hydroxyethylcellulose, sodium carboxymethylcellulose, polyvinyl alcohol, polyvinyl-pyrrolidone, polyacrylates, polyacrylamide, dextran, gellan gum, poloxamer, calcium polycarbophil or cellulose acetate phthalate. In a preferred embodiment of the present invention a preferred extender for the present formulations is a polymer such as polyvinylpyrrolidone, or a derivative thereof, having a mean molecular weight from about 10, to about 700,000 in a range from 0.5% to 15.0% by weight, preferably from 1% to 1.5% by weight, and may also include a cross-linking compound that will create ionic or covalent cross-linking of the polymer chains.

Polyvinylpyrrolidone (also called povidone or PVP), e.g., a linear polymer l-vinyl-2-pyrrolidone, extends muco-cilia clearance times of nasal spray compositions, particularly when an effective amount of a cross-linker such as short to medium chain polyethylene glycol (PEG) chains are present. Such polymers are commercially available as a series of products having mean molecular weights ranging from about 10,000 to about 700,000, and a single average molecular weight composition may be utilized in the present invention, or a mixture of polymers having differing average molecular weights. The PVP K-30 and PVK K90 compositions, as described below, are preferred average molecular weights for the present formulations. The various products marketed according to average molecular weights are designated K-values (i.e., K-15, K-30, K60, and K-90) as described below; e.g. GAF Corporation supplies PVP having the following K-values:

| K-Value | Average Molecular Weight |
|---|---|
| 15 | about 10,000 |
| 30 | about 40,000 |
| 60 | about 160,000 |
| 90 | about 360,000 |

The effect of polyvinylpyrrolidone on nasal muco-cilia clearance time is evaluated using a modified procedure as disclosed by Puchelle et al. (1981) Acta Otolarynogol 91:297-303. The procedure utilizes a concentrated sodium saccharin solution as the indicator. A 100 mcl dose of water-soluble polymer test solution can be sprayed into the nose. After spraying, a cotton swab saturated with saccharin solution is inserted into the nostril and wiped around the ostium depositing the saccharin onto the nasal mucosal lining. The clearance time is defined as the time for deposit of saccharin in the ostium to the time the saccharin was tasted in the back of the throat/mouth. Prior experimental results in the '483 patent (referenced earlier) has indicated that polyvinylpyrrolidone would extend nasal muco-cilia clearance times, e.g., incorporation of PVP K-90 at 0.25% can extend nasal muco-cilia clearance times from the normal 8 to 10 minutes to 20 to 25 minutes.

In addition to extenders as described above, water soluble polyethylene glycol (PEG) polymers can be utilized in the pharmaceutical compositions of this invention to promote moisturization of the nasal spray compositions in the nasal cavity, and possible cross-linking of PVP. Polyethylene glycol is a linear polymer formed by the addition reaction of ethylene glycol with ethylene oxide and such polymers are commercially available in average molecular weights ranging from about 200 to greater than 20,000. The commercially available grades of polyethylene glycol are marketed based on the average molecular weight, i.e. the grade nomenclature is identified with the molecular weight. For example, PEG 400 represents material with an average molecular weight of 400 and the material with an average molecular weight of 600 is known as PEG 600. PEG 200, 300, 400, and 600 are clear viscous liquids at room temperature; PEG 900, 1000, 1450, 3350, 4500 and 8000 are white, waxy solids. Preferred polyethylene glycols for the compositions of this invention are the short to medium chain PEG polymers such as PEG 400 to PEG 3350, and the most preferred polyethylene glycol is PEG 1450. The amount of polyethylene glycol that can optionally be present in the compositions of this invention is from about 0.00 to 15.0% by weight/volume of the total composition. Ranges of 0.5% to 10% by weight/volume of the total composition are particularly suitable and a range of 2.5 to 5% by weight/volume is most preferable.

The compositions of the present invention may optionally contain an aromatic alcohol selected from the group consisting of benzyl alcohol and phenyl ethyl alcohol, for example. The amount of aromatic alcohol optionally present in the composition can be from about 0 to 5.00% by weight/volume of the total composition. Ranges of 0.20-3.00% by weight/volume of the total composition are particularly suitable, and a range of 0.25 to 1.00% by weight/volume of the total composition being most preferable.

The compositions of the present invention may optionally contain other moisturizing agents. Examples of moisturizing agents useful in the compositions of this invention include propylene glycol, glycerin and the like. Mixtures of such moisturizing agents are also useful in the compositions. The amount of moisturizing agent optionally present in the composition according to the invention is from about 0 to 10% by weight/volume of the total composition. Ranges of 1.00 to 4.00% by weight/volume of the total composition are particularly suitable, and a range of 1.5 to 3.50% by weight/volume of the total composition being most preferable.

The compositions of the present invention may optionally contain a pharmaceutically acceptable antioxidant, e.g. disodium EDTA or a vitamin C/citrate buffer. The amount of antioxidant optionally present in the composition is from about 0 to 0.10% by weight/volume of the total composition. Ranges of 0.01 to 0.05% by weight/volume of the total composition are particularly suitable, and a range of 0.015 to 0.030% by weight/volume of the total composition being most preferable.

The compositions of the present invention can optionally contain at least one antimicrobial preservative in the range of 0.001% to about 0.3% by weight/volume of the composition. Examples of such antimicrobial preservatives are benzalkonium chloride, cetylpyridinium chloride/bromide, chlorobutanol, chlorhexidine acetate, chlorhexidine HCl, chlorhexidine digluconate, chlorocresol, methylparaben, propylparaben, butylparaben, phenoxyethanol, phenylmercury salts, sorbic acid, thiomersal. A preferred preservative which functions as an antimicrobial agent includes the commercially available preservative, benzalkonium chloride in the range of about 0.02 to about 0.025% by weight/volume. In addition to being preservatives, stronger antibiotics may be added at therapeutic levels well known in the art, if treatment of a patient with an antibiotic is indicated.

The compositions of the present invention can also optionally include pharmaceutically acceptable buffers sufficient to adjust and maintain the pH of the compositions of the present invention in the range of about 4.0 to about 8.0, preferably about 5.5 to about 7.0 and 6.25 to 6.75 being most preferable. Typically suitable buffers include citrate, phosphate, tromethamine, glycine, borate or acetate salts, which can also be derived from substances of the type such as citric acid, primary or secondary sodium phosphate, glycine, boric acid, sodium tetraborate, acetic acid and sodium acetate. Further excipients such as hydrochloric acid or sodium hydroxide can also be used for pH adjustment.

Moreover, suitable excipients cam be used for adjusting the tonicity or osmolality, such as sodium chloride, potassium chloride, mannitol, glucose, sorbitol, glycerol or propylene glycol in concentrations of from about 0.1 to about 10 weight percent.

The nasal aqueous and dry compositions of the present invention can be manufactured in a conventional manner by thoroughly mixing the ingredients at ambient or elevated temperatures in order to achieve solubility of ingredients where appropriate, and optional evaporation of the solvent system where dry components are desired, or by simply mixing dry ingredients. The formulations of the present invention may take any form suitable for delivering the active agents to a patient. For example, the formulations may be in the form of a dry powder, aerosol or liquid.

C. Dry Powder Formulations

The dry powder formulations as described herein include, at a minimum, both the topical decongestant and corticosteroid and a carrier. Such dry powder formulations can be administered via nasal inhalation to a patient without the benefit of a lot of carrier, but effective amounts of a sugar carrier or polymer may coat the granules to either aid or slow absorption. Preferably, dry powder formulations that do not include a large amount of carrier (or coated granules) are administered with the aid of dry powder inhaler types that are well known in the art.

Preferably, however, the dry powder formulations described herein include effective amounts one or more pharmaceutically acceptable carriers. Although any carrier suitable for nasal inhalation may be used, pharmaceutical sugars are particularly preferred for use as carriers in the present invention. Preferred pharmaceutical sugars include those selected from the group consisting of fructose, galactose, glucose, lactitol, lactose, maltitol, maltose, mannitol, melezitose, myoinositol, palatinite, raffinose, stachyose, sucrose, trehalose, xylitol, hydrates thereof, and combinations of any of the foregoing. It is particularly preferred, however, that lactose, e.g., lactose U.S.P., serves as the carrier in the present invention when the formulation is a dry powder.

Once selected, each active agent or the active agents in combination are blended to form a substantially homogeneous powder mixture. Techniques involved with the preparation of such powders are well known in the art. Briefly stated, however, the preparation generally includes the steps of reducing the particle size of each active agent (again, alone or in combination), and blending. Of course, reducing the particle size of each active agent is not required when a commercially available product having a suitable particle size is used. Techniques for reducing the particle size include, for example, using mills such as an air-jet mill or a ball mill. The active agents should have a particle size diameter of between about 0.1 μm to about 65 μm for pulmonary administration. It is preferred that the active agent particles are about 1 μm to about 10 μm, more preferably about 2 μm to about 5 μm in diameter.

Similarly, the particle size of the remaining components, e.g., carrier, excipient, etc., must be controlled as well. The same techniques described above for reducing the particle size of active agents may be used to reduce the particle size of the remaining components. Again, such techniques are not required when the component is available commercially in the desired particle size range. Preferably, the remaining components, particularly the carrier, have a particle size from about 30 μm to about 100 μm in diameter, with sizes from about 30 μm to about 70 μm most preferred.

For any given particle size range, it is preferred that at least about 60%, more preferably at least about 70%, still more preferably at least about 85%, of the stated particles have a size within the stated or given range. It is most preferred, however that at least about 90% of the particles have the size in the stated or given range. For example, when a component is stated to have a particle size less than 10 μm, it is most preferred that at least 90% of the particles of that component have a particle size of less than 10 μm.

As previously stated, some components of the formulation may be commercially available in the desired particle size range. For example, a preferred lactose product for use in some embodiments of the present invention is the PHARMATOSE™ 325 brand of lactose monohydrate available from DMV International, Veghel, The Netherlands. According to the manufacturer, 100% of the lactose particles have a particle size of less than 100 μm, and only 5 to 10% of the particles have a particle size of less than 32 μm. Furthermore, a minimum of 70% of the lactose particles are stated to have a particle size of less than 63 μm. Advantageously, particle size manipulation steps are avoided when components are commercially available in the desired particle size range.

Preferably, the particle size reduction of the active agents and the particle size reduction of the remaining components are carried out separately. In this way, it is possible to provide a formulation in which the particle size of the active agents is smaller than the particle size of, for example, the carrier. The advantage of such a formulation is that the active agents penetrate deeply into the pulmonary tract while the carrier (having a relatively larger particle size) is retained in the upper airways.

Conventional blending techniques known to those skilled in the art may be used for combining active agents or for combining the active agents with the carrier and/or remaining components. Such blending techniques include passing the combined powders through a sifter or blending, for example, the active agents and carrier in a powder blender such as a “double cone” blender or a “V-blender.” No matter which technique is employed, however, it is necessary that the resulting powder is a substantially homogeneous mixture. Typically, the active agents will make up from about 0.01% to about 99% of the total formulation, preferably from about 0.05% to 50% of the total formulation by weight.

After blending, the powder formulation may, if desired, be portioned and/or otherwise processed into unit dose quantities, e.g., portioned into unit dose quantities and individually placed within a dosage form or drug delivery system. Alternatively, the powder formulation may be loaded into a dosage form or drug delivery device and not “metered out” into unit doses until used. Although any dosage form that contains a unit dose of the formulation is acceptable, capsules are preferred. The capsule material may be either hard or soft, and, as will be appreciated by those skilled in the art, typically comprises a water-soluble compound such as gelatin, starch or a cellulosic material. Preferably, the capsules are composed of a cellulosic material, e.g., hydroxypropyl methylcellulose (HPMC). The capsules may be sealed, such as with gelatin bands or the like. See, for example, Remington. The Science and Practice of Pharmacy, supra, which describes materials and methods for preparing encapsulated pharmaceuticals. Thus, each capsule or dosage form will typically contain a therapeutically effective dose of each active agent.

Alternatively, the dosage forms may contain less than a therapeutically effective dose in which case administration of two or more dosage forms would be required to achieve the therapeutically effective dose.

D. Aerosol Formulations

The formulations of the present invention may also take the form of an aerosol composition for nasal or sinusoidal inhalation. Aerosol formulations are known to those-skilled in the art and are described in Remington: The Science and Practice of Pharmacy, supra. Briefly, the aerosol formulation of the invention is either a solution aerosol in which the active agents are soluble in the carrier (e.g., propellant) and optional solvent or a dispersion aerosol in which the active agents are suspended or dispersed throughout the carrier and optional solvent. It is preferred that the aerosol formulations of the invention are in the form of a dispersion aerosol.

The carrier in the aerosol formulations of the invention is generally a propellant, usually a compressed gas, e.g., air, nitrogen, nitrous oxide, and CO₂, a mixture of compressed gases, a liquefied gas or a mixture of liquefied gases. A mixture of propellants, when present in the formulations, may be comprised of two, three, four or propellants. Preferred mixtures of propellants, however, comprise only two propellants. Any propellant used in the art of preparing aerosol formulations may be used.

Typically, the propellant is a chlorofluorocarbon, a hydrochlorofluorocarbon, a hydrogen-containing fluorocarbon, a perfluorocarbon, a hydrocarbon or a mixture thereof. Preferably, the propellant is a hydrochlorofluorocarbon, a hydrogen-containing fluorocarbon, a perfluorocarbon or a mixture thereof.

Preferred chlorofluorocarbons include dichlorotetrafluoroethane (e.g., CClF₂CClF₂ and CCl₂FCF₃), trichloromonofluoromethane, dichlorodifluoromethane, chloropentafluoroethane, and mixtures thereof. Preferred hydrochlorofluorocarbons include monochlorodifluoromethane, monochlorodifluoroethane (e.g., 1-chloro-1,1-difluoroethane), and mixtures thereof. Preferred hydrogen-containing fluorocarbons include C₁₋₄ hydrogen-containing fluorocarbons such as CHF₂CHF₂, 1,1,1,2-tetrafluoroethane (HFA-134a), difluoroethane (e.g., 1,1-difluoroethane), 1,1,1,2,3,3,3-heptafluoropropane (HFA-227), and mixtures thereof. Preferred perfluorocarbons include CF₃CF₃, CF₃CF₂CF₃, octafluorocyclobutane, and mixtures thereof. Preferred hydrocarbons include propane, isobutane, n-butane, dimethyl ether, and mixtures thereof. Most preferably, the propellant is selected from the group consisting of difluoroethane, CHF₂CHF₂, 1,1,1 ,2-tetrafluoroethane, 1,1,1,2,3,3,3-heptafluoropropane, CF₃CF₃, CF₃CF₂CF₃, octafluorocyclobutane, and mixtures of any of the foregoing.

As will be appreciated by one skilled in the art, the aerosol formulations of the invention may include effective amounts of one or more carriers or excipients. For example, the aerosol formulations may contain: a solvent (e.g., water, ethanol and mixtures thereof) for increasing the solubility of the active agent; an antioxidant (e.g., ascorbic acid) for inhibiting oxidative degradation of the active agents; a dispersing agent (e.g., sorbitan trioleate, oleyl alcohol, oleic acid, lecithin, e.g., soya lecithin, corn oil, or combinations thereof) for preventing agglomeration of particles; and/or a lubricant (e.g., isopropyl myristate) for providing slippage between particles and lubricating the components, e.g., the valve and spring, of the inhaler. In some instances, the propellant is sufficient to suspend and deliver the drug with no excipient being added.

As described with respect to dry powder formulations in Section C, the particle size released from aerosol formulations must be appropriate for nasal and sinusoidal administration. Solution aerosols inherently produce small particles upon actuation of the inhaler given that the active agent is expelled along with the carrier, i.e., propellant, solution as it evaporates. Consequently, solution aerosols produce sufficiently small particles, e.g., within a range of about 0.1 μm to about 65 μm, of active agents upon administration. In contrast, dispersion aerosols contain undissolved active agents in which particle size remains constant, i.e., the size of the particles in the dispersion aerosol remains unchanged as the active agent is delivered to the patient. Thus, the active agents must have an appropriate particle size before being formulated into a dispersion aerosol. Consequently, methods of reducing the particle size of the active agents for the dry powder formulations described above are equally applicable for preparing active agents with an appropriate particle size in a dispersion aerosol. Furthermore, the same ranges of particle sizes preferred for the dry powder formulations are equally applicable for dispersion aerosols.

The aerosol formulation may be prepared by employing a cold filling process. Initially, the components of the aerosol formulation and an aerosol container are cooled, e.g., to about -40° C., such that the carrier, i.e., propellant, is a liquid. All components except for the carrier are placed into the aerosol container. Thereafter, the carrier is added, the components mixed, and a valve assembly inserted into place. The valve assembly is then crimped such that the container is airtight. Thereafter, the container and formulation contained therein are allowed to return to ambient temperature.

As an alternative to the cold filling process, the aerosol formulation may be prepared by transfer of a carrier from a bulk container. In such a process, the components except for the carrier are initially placed into an empty aerosol container. A valve assembly is then inserted and crimped into place. The carrier, under pressure and in liquid form, is metered through the valve assembly from a bulk container or tank of carrier. The container housing the formulation is checked to ensure that the pressurized contents do not leak.

For both of these methods of preparing the aerosol formulations, the active agents generally represent from about 0.1 wt % to about 40 wt % of the total formulation. It is preferred, however, that the active agents represent about 2 wt % to about 20 wt % of the total formulation, with 5 wt % to about 15 wt % being most preferred.

E. Liquid Formulations

The formulations of the present invention may also take the form of a liquid composition for nasal/sinusoidal administration by spraying or inhalation, including compositions formulated for metered dosage application. Liquid formulations are well known in the art. See, for example, Remington: The Science and Practice of Pharmacy, supra. It is preferred that the liquid is an aqueous suspension, although aqueous solutions may be used as well. The liquid formulations include one or more carriers in addition to the active agents (see the details set forth above). Generally, the carrier is a sodium chloride solution having a concentration such that the formulation is isotonic relative to normal body fluid. In addition to the carrier, the liquid formulations may contain water and/or excipients including an antimicrobial preservative (e.g., benzalkonium chloride, benzethonium chloride, chlorobutanol, phenylethyl alcohol, thimerosal and combinations thereof), a buffering agent (e.g., citric acid, potassium metaphosphate, potassium phosphate, sodium acetate, sodium citrate, and combinations thereof), a surfactant (e.g., polysorbate 80, sodium lauryl sulfate, sorbitan monopalmitate and combinations thereof), and/or a suspending agent (e.g., agar, bentonite, microcrystalline cellulose, sodium carboxymethylcellulose, hydroxypropyl methylcellulose, tragacanth, veegum and combinations thereof). Combining the components followed by conventional mixing results in a liquid formulation suitable for inhalation. Typically, the active agents will make up from about 0.01% to about 40% of the total formulation.

### III. Utility and Administration

The invention provides a method for treating a patient suffering from or prone to a condition, disease or disorder that is responsive to treatment with a topically administrable decongestant/corticosteroid combination, comprising nasal administration to a patient a pharmaceutical formulation for nasal drug administration, wherein the formulation comprises: a therapeutically effective amount of a topically administrable decongestant; a therapeutically effective amount of a topically administrable corticosteroid; and a pharmaceutically acceptable carrier suitable for nasal drug administration. For example, the method is particularly advantageous for the long-term treatment of patients suffering from chronic obstructive nasal congestion and/or OSA, and the like.

The formulations and methods as described herein have many advantages over conventional formulations. Because the pharmaceutical formulation combines these two active agents, patients can receive the benefits of long-term nasal decongestant use without substantially diminished decongestant activity and by reduction or elimination of addictive tolerance or excessive rebound upon cessation of treatment, or when skipping days of treatment. Furthermore, particular ingredients may have additional advantages when present in the formulation. For example, an initial formulation including one or more of antihistamines, antibiotics, lipoxygenase inhibitors, leukotriene antagonists, FLAP antagonists, and the like, may be utilized to gain control over existing secondary conditions or symptoms, and then a daily maintenance formulation may be utilized to treat or prevent chronic obstructive nasal congestion or OSA. Finally, the patient may only need to administer the formulation on an “as-needed” basis, such as just before or during exposure to environmental conditions that will exacerbate the underlying condition. Consequently, daily administration may not be necessary, but is still available to a patient who needs daily administration to control an underlying condition.

Patients' compliance for taking medications is directly related to the rapid relief of symptoms they experience from using a drug or combination of drugs. Nasal steroids provide excellent relief of the rhinorrhea, sneezing, nasal congestion and itching associated with an allergic or non-allergic rhinosinusitis, however, their onset of action is very slow taking anywhere from 48 to 72 hours. Their full benefit may not occur for 96 hours. There are several factors that contribute to this slow onset of action. The first is the edema of the nasal membranes that interferes with the absorption of this drug. The second is the transudate that washes the drug off the surface of the mucosa before it has a chance to penetrate the nasal membrane. Because of the very slow relief of symptoms patients quickly stop using nasal steroids even though they are one of the most effective treatment modalities. A new approach for the treatment of allergic and non-allergic rhinitis, and related diseases is therefore required. The following is a description of a combination of two different topical nasal medications that appears to be very effective in providing immediate benefit, provide for long-term treatment, and increase patient compliance.

A preferred combination according to the invention includes triamcinolone acetonide as the corticosteroid (such compositions may utilize a commercially available corticosteroid composition, such as Nasacort AQ, as a starting material) and oxymetazoline 0.05% (such compositions may utilize a commercially available nasal congestant composition, such as Nostrilla, as a starting material). Triamcinolone acetonide is available commercially as a micronized suspension in an aqueous medium, and the oxymetazoline 0.05% is a commercially available as an aqueous-based nasally administrable decongestant. The oxymetazoline 0.05%/triamcinolone acetonide solutions are administered separately or mixed and administered in a ratio of from 6:1 to 1:1, preferably 4:1 to 1:1 and more preferably in a 3:1 to 2:1 ratio.

The precise mechanism of action of topical corticosteroids is not known, since corticosteroids have a wide range of inhibitory activities against multiple cell types and mediators. However, the end result of these corticosteroid inhibitory activities is to reduce inflammation, i.e., swelling, redness and mucous secretion in the nasal area. Oxymetazoline is a synthetic sympathomimetic amine. It primarily acts upon the vascular system in a localized tissue area by shrinking swollen nasal mucous membranes and thereby reducing nasal congestion and rhinorrhea.

Surprisingly, combining the slow-acting nasal steroid (e.g., triamcinolone acetonide) with a rapid-acting sympathomimetic nasal decongestant (e.g., oxymetazoline 0.5%) provides the patient with immediate symptomatic relief of their nasal stuffiness and rhinorrhea followed by prolonged symptomatic relief afforded by the nasal steroid. The oxymetazoline causes a vasoconstriction of the blood vessels to diminish the nasal mucosal edema, prevent epistaxis and abolish the watery transudate that inevitably accompanies the edema. These actions of the oxymetazoline increase the efficacy of the steroid and reduce the amount of steroid needed in the combination solution or combination administration. Surprisingly, by adding the anti-inflammatory (the corticosteroid) to the decongestant or co-administering it with the decongestant, the potential for developing rhinitis medicamentosa (inflammation of the nasal mucous membranes) and rebound nasal congestion caused by the decongestant (e.g., oxymetazoline) are avoided. Therefore, both short-term and long-term compliance for taking this medication is increased because the patient experiences almost immediate relief of their symptoms, and avoids rebound nasal congestion when the combination is administered over a long period of time.

Additional advantages result when the formulations are administered via the preferred dry powder inhalers described in section IV, infra. Such dry powder inhalers assure that patients, particularly those patients that have traditionally had trouble using inhalers such as children or the elderly, obtain the complete dose. Even medical personnel who are responsible for monitoring and instructing patients in optimal inhaler use lack the rudimentary skills associated with MDIs. See Hanania et al. (1994) Chest 105(1):111-116. Administration of the complete dose is ensured with these preferred dry powder inhalers since little effort in inhalation is required in order to completely deliver all of the dose to the lungs. This is in contrast to, for example, metered-dose inhalers with which patients must coordinate the actuation of the inhaler with a deep and prolonged inhalation to ensure that the entire dose is received. As a result of the foregoing advantages, the dry powder inhalers described herein may be efficient in delivering the present formulations in reduced dosages, i.e., 5% to 15% less than the dose used in conventional devices.

The actual amount of each active agent in the formulation will, of course, depend upon the age, weight, and general condition of the subject, the severity of the condition being treated, and the judgment of the prescribing physician. Therapeutically effective amounts are known to those skilled in the art and/or are described in the pertinent reference texts and literature. An effective amount of the formulation may be administered with a single administration, e.g., serially administration of the contents of a single capsule containing a therapeutically effective amount of the formulation via a dry powder inhaler or a single actuation of an aerosol inhaler designed to deliver a therapeutically effective amount of the formulation. Alternatively, a patient can obtain an effective amount of the formulation by, for example, administering multiple doses, e.g., serially administering the contents of multiple capsules containing the formulation via a dry powder inhaler.

Furthermore, the actual amount of each active agent will also depend on particular “synergies” between the two active agents, and the effective amount of a particular agent. That is, certain combinations and/or ratios of the decongestant/corticosteroid combinations described herein provide enhanced treatment of a particular condition.

For the decongestant, for example, the formulation will be prepared such that each dose (or administration) of the formulation will deliver the decongestant in a therapeutically effective amount, typically in the range of about 1 μg to about 1500 fig, preferably from about 25 μg to about 100 μg.

The formulations may be administered in a variety of dosing regimens including: as-needed administration; one, two, three or four administrations once daily; one, two, three or four administrations twice daily; one, two, three or four administrations three times daily; and one, two, three or four administrations four times daily. Generally, however, the total daily dose of the decongestant administered in the combination formulation of the present invention should not exceed about the FDA approved maximum dose for a particular decongestant (as currently approved for solo administration). Likewise, the total daily dose for a corticosteroid administered in the combination formulation should not exceed the FDA approved maximum dose for a particular corticosteroid (as currently approved for solo administration).

The formulations of the invention may be administered via nasal inhalation or by use of a propellant system to deliver the formulation to the nasal passages and/or nasal sinuses. The inhaled formulation progressively comes into contact with the air passages of the nasal passages, cranial sinuses and throat area, e.g., the upper respiratory tract. During nasal inhalation the patient inhales the formulation through the nares, preferably one at a time. For example, the formulation may be administered via a pump spray in which the patient administers a spray into the left nare followed by administration into the right nare. Nasal administration provides an added benefit of relieving nasal congestion (if present) in that the corticosteroid is placed in contact with nasal tissue.

The formulations described above have been formulated to obtain the desired daily dosages by administration of the formulations once or twice daily, as needed. Of course, the individual daily doses necessary will vary depending upon a particular individual and the severity of their condition, but tailoring a daily dosage to a particular individual is well within the ordinary skill of a practicing physician or pharmacist in this field. Particular modes of nasal administration and devices for delivering meter doses by powder, spray, aerosol, or other liquid application methods are well known in this field to the ordinary practitioner.

It is to be understood that while the invention has been described in conjunction with the preferred specific embodiments thereof, that the foregoing description as well as the examples that follow are intended to illustrate and not limit the scope of the invention. Other aspects, advantages and modifications within the scope of the invention will be apparent to those skilled in the art to which the invention pertains.

All patents, patent applications, and publications mentioned herein are hereby incorporated by reference in their entireties.

### EXPERIMENTAL

The practice of the present invention will employ, unless otherwise indicated, conventional techniques of pharmaceutical formulation and the like, which are within the skill of the art. Such techniques are explained fully in the literature.

In the following examples, efforts have been made to ensure accuracy with respect to numbers used (e.g., amounts, temperature, etc.) but some experimental error and deviation should be accounted for. Unless indicated otherwise, temperature is in degrees C and pressure is at or near atmospheric. All reagents were obtained commercially unless otherwise indicated.

### EXAMPLE 1

### Nasal Spray or Drops Containing Oxymetazoline and Mometasone

Add 4.5 Kg of purified water into a suitable stirrer-equipped container. Add 2.5 g of oxymetazoline hydrochloride (decongestant), 2.5 g of mometasone furoate monohydrate (corticosteroid), 5 g of hydroxymethylcellulose,2.5 g Sodium edetate, 0.625 g benzalkonium chloride, sorbitol solution 70% 333.3 g. Add enough purified water to bring the volume to 4.95 L, and then adjust the solution to a pH of 6.0 by adding pharmaceutical grade 1 N sodium hydroxide. Add more purified water with stirring, in an amount sufficient to give a final solution volume of five liters (5 L). Filter the solution through a membrane filter having a pore size of 0.2 μM and dispense into pump spray bottles (such as the spray bottle disclosed in the Schering Corporation Industrial Design Deposit DM/026304, registered Jun. 1, 1993). Such a solution (about 0.05% decongestant and about 0.05% corticosteroids) and metering device is expected provide the appropriate dose (one or two sprays into each nare of the nasal passages) for once or twice daily administration.

TABLE 1

| Component | amount per 100 mL |
|---|---|
| Oxymetazoline HCl | 0.0500 g |
| Momethasone furoate (anhydrous) | 0.0500 g |
| Sorbitol Solution 70% | 6.666 g |
| Benzalkonium chloride | 0.0125 g |
| Sodium edetate | 0.0500 g |
| Sodium hydroxide | q.s. to pH 6.0 |
| Purified water | to 100 mL volume |

### EXAMPLE 2

### Nasal Spray or Drops Containing Oxymetazoline and Beclomethasone

Add 4.5 Kg of purified water into a suitable stirrer-equipped container. Add 2.5 g of oxymetazoline hydrochloride (decongestant), 2.5 g of beclomethasone dipropionate (corticosteroid), 5 g of hydroxymethylcellulose, 2.5 g Sodium edetate, 0.625 g benzalkonium chloride, sorbitol solution 70% 333.3 g. Add enough purified water to bring the volume to 4.95 L, and then adjust the solution to a pH of 6.0 by adding pharmaceutical grade 1 N sodium hydroxide. Add more purified water with stirring, in an amount sufficient to give a final solution volume of five liters (5 L). Filter the solution through a membrane filter having a pore size of 0.2 μM and dispense into pump spray bottles (such as the spray bottle disclosed in the Schering Corporation Industrial Design Deposit DM/026304, registered Jun. 1, 1993). Such a solution (about 0.05% decongestant and about 0.05% corticosteroids) and metering device is expected to provide the appropriate dose (one or two sprays into each nare of the nasal passages) for once or twice daily administration.

TABLE 2

| Component | amount per 100 mL |
|---|---|
| Oxymetazoline HCl | 0.0500 g |
| Beclomethasone dipropionate | 0.0500 g |
| Sorbitol Solution 70% | 6.666 g |
| Benzalkonium chloride | 0.0125 g |
| Sodium edetate | 0.0500 g |
| Sodium hydroxide | q.s. to pH 6.0 |
| Purified water | to 100 mL volume |

### EXAMPLE 3

### Nasal spray or drops Containing Oxymetazoline, Beclomethasone and Chlorpheniramine

Add 4.5 Kg of purified water into a suitable stirrer-equipped container. Add 2.5 g of oxymetazoline hydrochloride (decongestant), 2.5 g of beclomethasone dipropionate (corticosteroid), 25 g of Chlorpheniramine maleate (antihistamine), 5 g of hydroxy-methylcellulose, 2.5 g Sodium edetate, 0.625 g benzalkonium chloride, sorbitol solution 70% 333.3 g. Add enough purified water to bring the volume to 4.9 L, and then adjust the solution to a pH of 6.0 by adding pharmaceutical grade 1 N sodium hydroxide. Add more purified water with stirring, in an amount sufficient to give a final solution volume of five liters (5 L). Filter the solution through a membrane filter having a pore size of 0.2 μM and dispense into pump spray bottles (such as the spray bottle disclosed in the Schering Corporation Industrial Design Deposit DM/026304, registered Jun. 1, 1993). Such a solution (about 0.05% decongestant, about 0.05 % corticosteroids, and about 0.25% antihistamine) and metering device is expected to provide the appropriate dose (one or two sprays into each nare of the nasal passages) for once or twice daily administration, when an antihistamine is desired.

TABLE 3

| Component | amount per 100 mL |
|---|---|
| Oxymetazoline HCl | 0.0500 g |
| Beclomethasone dipropionate | 0.0500 g |
| Chlorpheniramine maleate | 0.2500 g |
| Sorbitol Solution 70% | 6.666 g |
| Benzalkonium chloride | 0.0125 g |
| Sodium edetate | 0.0500 g |
| Sodium hydroxide | q.s. to pH 6.0 |
| Purified water | to 100 mL volume |

### EXAMPLE 4

### Nasal Spray or Drops Containing Oxymetazoline, Beclomethasone and Azelastine

Add 3.50 Kg of purified water into a suitable stirrer-equipped container and heat to 50° C. Individually add the following ingredients to water with stirring, while maintaining the above temperature: 1.0 g sodium edetate, 4.98 g sodium phosphate dibasic, 27.6 g sodium phosphate monobasic, 12.5 g PVP K-90, 50 g PVP K-30 and 12.5 g benzyl alcohol. Stir the mixture for 5 minutes after adding each of the individual ingredients. With continued mixing individually add the water soluble polymers 12.5 g PVP K-90, 50 g PVP K-30, and 125 g of PEG1450, and stir the mixture for 5 minutes after the addition of each polymer. Individually add with mixing each of 2.5 g of oxymetazoline hydrochloride (decongestant), 2.5 g of beclomethasone dipropionate (corticosteroid) and 5.0 g of azelastine hydrochloride (antihistamine), and stir the mixture for 5 minutes after the addition of each ingredient. With mixing, add 1.0 g benzalkonium chloride (17% solution) and mix for at least 5 minutes after the addition is complete. Cool the mixture to 30° C. with stirring and then add more purified water with stirring, in an amount sufficient to give a final solution volume of five liters (5 L) and mix until a uniform mixture is obtained. Filter the solution utilizing conventional filtering equipment and dispense into pump spray bottles (such as the spray bottle disclosed in the Schering Corporation Industrial Design Deposit DM/026304, registered Jun. 1, 1993). Such a solution (about 0.05% decongestant, about 0.05% corticosteroids, and about 0.10% antihistamine) and metering device is expected to provide the appropriate dose (one or two sprays into each nare of the nasal passages) for once or twice daily administration, when an antihistamine is desired.

TABLE 4

| Component | amount per 100 mL |
|---|---|
| Oxymetazoline HCl | 0.0500 g |
| Beclomethasone dipropionate | 0.0500 g |
| Azelastine HCl | 0.2500 g |
| Benzyl Alcohol | 0.2500 g |
| Benzalkonium chloride (17%) | 0.0200 g |
| Disodium EDTA | 0.0200 g |
| Sodium Phosphate Dibasic | 0.0975 g |
| Sodium Phosphate Monobasic | 0.5525 g |
| PEG 1450 | 2.500 g |
| PVP K-30 | 1.000 g |
| PVP K-90 | 0.2500 g |
| Purified water | to 100 mL volume |

### EXAMPLE 5

### Sustained Delivery Nasal Spray Containing Oxymetazoline and Beclomethasone

The same procedures were followed with the same proportions as in Example 4, above, except that the antihistamine azelastine hydrochloride was omitted and additional water was added to yield the 5 L solution. Such a solution (about 0.05% decongestant and about 0.05 % corticosteroids) and metering device will provide the appropriate dose (one or two sprays into each nare of the nasal passages) for once or twice daily administration, when an antihistamine is desired, and will provide a sustained/delayed/gradual release type composition for administration.

TABLE 5

| Component | amount per 100 mL |
|---|---|
| Oxymetazoline HCl | 0.0500 g |
| Beclomethasone dipropionate | 0.0500 g |
| Benzyl Alcohol | 0.2500 g |
| Benzalkonium chloride (17%) | 0.0200 g |
| Disodium EDTA | 0.0200 g |
| Sodium Phosphate Dibasic | 0.0975 g |
| Sodium Phosphate Monobasic | 0.5525 g |
| PEG 1450 | 2.500 g |
| PVP K-30 | 1.000 g |
| PVP K-90 | 0.2500 g |
| Purified water | to 100 mL volume |

### EXAMPLE 6

Oxymetazoline hydrochloride (10.0 mg), mometasone furoate (10 mg, anhydrous) and 2000 mg of lactose are blended using conventional blending techniques to form a dry pharmaceutical formulation. The dry pharmaceutical formulation is then placed, in equal portions, into 100 capsules (capsule size 4). The amount of each component of the formulation per capsule is presented in Table 6. Such capsules may be pierced to dispense a unit dose into a nasal inhaler for such dry powder administration. Other dispensing devices may be utilized without forming capsules, for example one may wish to use a dry volume dispensing device that can be rotated to dispense from a larger chamber into a smaller chamber a measured amount of the dry formulation, followed by closing off the larger chamber by rotating or other means before administering a unit dose from the smaller chamber (or from a third chamber connected therewith).

TABLE 6

| Component | amount per capsule |
|---|---|
| Oxametazoline HCl | 100 μg |
| Mometasone furoate (anhydrous) | 100 μg |
| Lactose | 20.00 mg |

### EXAMPLE 7

The capsules made in Examples 6 (or similar capsules containing only 50% the dosage amount illustrated in Example 6) are placed in the dry powder inhaler as described in U.S. Pat. Nos. 5,673,686 to Villax et al. and 5,881,721 to Bunce et al., except that the inhaler design and inhaler tip has been modified for intranasal administration instead of administration by mouth, i.e., the inhaler tip has been adapted to fit inside the nares of the nasal passages. Once the capsule has been properly aligned and pierced in the inhaler, a patient in need of administration of the compositions according to the present invention holds on nostril closed and the nasal tip of the inhaler into his other nostril nare and inhales strongly through the open nostril to inhale the formulation (snorts it). The inhalation is expected to cause the formulation to exit the pierced capsule and travel into the patient's nasal and sinus passages of the upper respirator system.

### EXAMPLE 8

### Nasal Spray or Drops Containing Oxymetazoline and Triamcinolone Acetonide

A commercially available 0.05% by weight aqueous solution of Oxymetazoline (e.g. nostrilla® was obtained and a commercially available 0.05% by weight aqueous solution of Triamcinolone Acetonide was obtained. The two aqueous solutions were mixed in 3:1 and 2:1 volume ratios of Oxymetazoline to Triamcinolone Acetonide, and then placed in a container that is typically used to administer a metered dose of Oxymetazoline as 2 or 3 sprays (such as the spay bottle disclosed in the Schering Corporation Industrial Design Deposit DM/026304, registered Jun. 1, 1993). These solutions were administered nasally every twelve hours as 3 to 4 spray to each nostril with such a metering device to provide a therapeutically effective dose of the composition.

What is claimed is:

1. A method for treating a patient suffering from a condition, disease or disorder that is responsive to treatment with a decongestant/corticosteroid combination, comprising nasally administering to the patient a pharmaceutical formulation for intranasal drug administration, wherein the formulation comprises:
a therapeutically effective amount of a topically administrable decongestant;
a therapeutically effective amount of a topically administrable corticosteroid; and
a pharmaceutically acceptable carrier that is suitable for nasal drug administration.

2. The method of claim 1, wherein the topically administrable decongestant is a sympathomimetic amine.

3. The method of claim 2 wherein the sympathomimetic amine is selected from the group consisting of: oxymetazoline, xylometazoline, naphazoline, tetrahydrozoline, phenylephrine, pharmaceutically acceptable salts thereof, and combinations of any of the foregoing.

4. The method of claim 1, wherein the topically administrable corticosteroid is selected from the group consisting of: beclomethasone, budesonide, flunisolide, fluticasone, flunidolone, mometasone, triamcinolone, dexamethasone, pharmaceutically acceptable salts and esters thereof.

5. The method of claim 3, wherein the topically administrable corticosteroid is selected from the group consisting of: beclomethasone, budesonide, flunisolide, fluticasone, flunidolone, mometasone, triamcinolone, dexamethasone, and pharmaceutically acceptable salts and esters thereof.

6. The method of claim 5, wherein the formulation further comprises a therapeutically effective amount of an additional topically administrable pharmaceutically active agent.

7. The method of claim 6, wherein the additional active agent is selected from an antibiotic, an anticholinergic, an antihistamine, a leukotriene D₄ antagonist, a 5-lipoxygenase inhibitor, a FLAP antagonist and combinations thereof.

8. The method of claim 7, wherein the additional active agent is an antihistamine.

9. The method of claim 8, wherein the antihistamine is a non-sedating type antihistamine.

10. The method of claim 1, wherein the patient is suffering from obstructive sleep apnea.

11. The method of claim 5, wherein the patient is suffering from obstructive sleep apnea.

12. The method of claim 10, wherein the formulation is administered at least once daily for a period greater than 14 days.

13. The method of claim 11, wherein the formulation is administered at least once daily for a period greater than 30 days.

14. The method of claim 1, wherein the patient is suffering from chronic nasal congestion.

15. The method of claim 5, wherein the patient is suffering from chronic nasal congestion.

16. The method of claim 14, wherein the formulation is administered at least once daily for a period greater than 14 days.

17. The method of claim 15, wherein the formulation is administered at least once daily for a period greater than 30 days.

18. The method of claim 1, wherein the formulation is administered in the form of a dry powder.

19. The method of claim 18, wherein the corticosteroid is mometasone or an ester thereof.

20. The method of claim 19, wherein the corticosteroid is a mometasone ester.

21. The method of claim 20, wherein the mometasone ester is anhydrous mometasone furoate.

22. The method of claim 20, wherein the mometasone ester is anhydrous mometasone furoate monohydrate.

23. The method of claim 18, wherein the carrier is fructose, galactose, glucose, lactitol, lactose, maltitol, maltose, mannitol, melezitose, myoinositol, palatinite, raffinose, stachyose, sucrose, trehalose, xylitol, hydrates thereof, or a combination of any of the foregoing.

24. The method of claim 23, wherein the carrier is lactose.

25. The method of claim 1, wherein the formulation is administered in the form of an aqueous solution.

26. The method of claim 25, wherein the carrier comprises a biocompatible hydrophilic polymer.

27. The method of claim 26, wherein the biocompatible hydrophilic polymer comprises polyvinylpyrrolidone or a derivative thereof.

28. The method of claim 27, wherein the polyvinylpyrrolidone polymer or a derivative thereof has an average molecular weight of about 10,000 to 360,000.

29. The method of claim 28, wherein the polyvinylpyrrolidone or polyvinylpyrrolidone derivative is present in the formulation in an amount from 0.50 to 15.00% by weight/volume of the aqueous carrier.

30. The method of claim 26, wherein the formulation further comprises a crosslinking agent capable of crosslinking chains of the biocompatible hydrophilic polymer.

31. The method of claim 30, wherein the crosslinking forms a polymeric structure having bioadhesive properties.

32. The method of claim 31, wherein the bioadhesive properties provide an extended therapeutic effect for an active agent present in the formulation.

33. The method of claim 31, wherein the biocompatible hydrophilic polymer is polyvinylpyrrolidone or a derivative thereof.

34. The method of claim 33, wherein the cross-linking agent comprises short to mid-length chains of polyethylene glycol.

35. The method of claim 1, wherein the corticosteroid represents approximately 0.01% to 1% by weight of the pharmaceutical formulation.

36. The method of claim 35, wherein the corticosteroid represents approximately 0.05% to 0.5% by weight.

37. The method of claim 36, wherein the corticosteroid represents approximately 0.05% to 0.1% by weight.

38. The method of claim 1, wherein the formulation is administered in a unit dosage form containing corticosteroid in an amount of approximately 10 to 100 micrograms.

39. The method of claim 1, wherein the decongestant represents approximately 0.001% to 0.2% by weight of the administered pharmnaceutical formulation.

40. The method of claim 39, wherein the decongestant represents approximately 0.01% to 0.1% by weight.

41. The method of claim 40, wherein the decongestant represents approximately 0.025 to 0.05% by weight.

42. The method claim 8, wherein the antihistamine represents approximately 0.001 to 2.0% by weight of the pharmaceutical formulation.

43. The method of claim 42, wherein the formulation is administered in the form of a liquid nasal spray or aerosol composition.

44. A pharmaceutical formulation for nasal drug administration, comprising:
a therapeutically effective amount of a topically administrable decongestant;
a therapeutically effective amount of a topically administrable corticosteroid; and
a pharmaceutically acceptable carrier that is suitable for nasal drug administration.

45. The formulation of claim 44, wherein the topically administrable decongestant is a sympathomimetic amine.

46. The formulation of claim 45, wherein the sympathomimetic amine is selected from the group consisting of: oxymetazoline, xylometazoline, naphazoline, phenylephrine, pharmaceutically acceptable salts thereof, and combinations of any of the foregoing.

47. The formulation of claim 44, wherein the topically administrable corticosteroid is selected from the group consisting of: beclomethasone, budesonide, flunisolide, fluticasone, flunidolone, mometasone, triamcinolone, dexamethasone, pharmaceutically acceptable salts and esters thereof.

48. The formulation of claim 45, wherein the topically administrable corticosteroid is selected from the group consisting of: beclomethasone, budesonide, flunisolide, fluticasone, flunidolone, mometasone, triamcinolone, dexamethasone, and pharmaceutically acceptable salts and esters thereof.

49. The formulation of claim 48, wherein the formulation further comprises a therapeutically effective amount of an additional topically administrable pharmaceutically active agent.

50. The formulation of claim 49, wherein the additional active agent is selected from the group consisting of: an antibiotic, an anticholinergic, an antihistamine, a leukotriene D₄ antagonist, a 5-lipoxygenase inhibitor, a FLAP antagonist and combinations thereof.

51. The formulation of claim 50, wherein the additional active agent is an antihistamine.

52. The formulation of claim 51, wherein the antihistamine is a non-sedating type antihistamine.

53. The formulation of claim 44, in a dry powder form.

54. The formulation of claim 53, wherein the corticosteroid is mometasone or an ester thereof.

55. The formulation of claim 54, wherein the corticosteroid is a mometasone ester.

56. The formulation of claim 55, wherein the mometasone ester is anhydrous mometasone furoate.

57. The formulation of claim 55, wherein the mometasone ester is anhydrous mometasone furoate monohydrate.

58. The formulation of claim 53, wherein the carrier is fructose, galactose, glucose, lactitol, lactose, maltitol, maltose, mannitol, melezitose, myoinositol, palatinite, raffinose, stachyose, sucrose, trehalose, xylitol, hydrates thereof, or a combination of any of the foregoing.

59. The formulation of claim 58, wherein the carrier is lactose.

60. The formulation of claim 44 in the form of an aqueous solution.

61. The formulation of claim 60, comprising a hydrophilic polymer.

62. The formulation of claim 61, wherein the hydrophilic polymer comprises polyvinylpyrrolidone or a derivative thereof.

63. The formulation of claim 62, wherein the polyvinylpyrrolidone polymer or a derivative flunisolide, fluticasone, flunidolone, mometasone, triamcinolone, dexamethasone, and thereof has an average molecular weight of about 10,000 to 360,000.

64. The formulation of claim 63, wherein the polyvinylpyrrolidone or polyvinylpyrrolidone derivative is present in the formulation in an amount from 0.50 to 15.00% by weight/volume of the aqueous carrier.

65. The formulation of claim 61, further comprising a cross-linking agent capable of crosslinking polymer chains.

66. The formulation of claim 65, wherein the crosslinking forms a polymeric structure having bioadhesive properties.

67. The formulation of claim 66, wherein the bioadhesive properties provide an extended therapeutic effect for an active agent present in the formulation.

68. The formulation of claim 60, wherein the corticosteroid represents approximately 0.01% to 1% by weight.

69. The formulation of claim 68, wherein the corticosteroid represents approximately 0.05% to 0.5% by weight.

70. The formulation of claim 69, wherein the corticosteroid represents approximately 0.05% to 0.1% by weight.

71. The formulation of claim 69, in a unit dosage form wherein corticosteroid is present in an amount of approximately 10 to 100 micrograms.

72. The formulation of claim 69, wherein the decongestant represents approximately 0.001% to 0.2% by weight.

73. The formulation of claim 69, wherein the decongestant represents approximately 0.01% to 0. 1% by weight.

74. The formulation of claim 73, wherein the corticosteroid represents approximately 0.025 to 0.05% by weight.

75. The formulation of claim 51, wherein the antihistamine represents approximately 0.001 to 2.0% by weight.

76. The formulation of claim 51, in the form of liquid nasal spray or aerosol composition.

77. An nasal administrable drug delivery device, comprising: a pharmaceutical formulation as described in claim 44, and a means for housing and dispensing unit dosages of the formulation into or through a nasal passage of a patient.

78. The drug delivery device of claim 77, comprising a dry powder inhaler, metered-dose inhaler, nebulizer or pump spray bottle.

79. The drug delivery device of claim 78, in the form of a dry powder inhaler.

80. A dry powder inhaler for orienting and positioning a capsule containing a pharmaceutical formulation to be administered via inhalation through a nasal opening, comprising:
a dispensing chamber containing a capsule of a dry powder pharmaceutical formulation as described in claim 44;
a tube for receiving the capsule to be oriented and dispensed;
a ramp surface extending substantially across the tube from one wall to an opposite wall thereof, and
an elongate dispensing passage having a diameter less than that of the tube and sized to receive the capsule only when the elongate axis of the capsule is generally parallel to the axis of the passage, the passage extending from an inlet end formed by an aperture in the ramp's surface to a dispensing outlet, the passage being adjacent to one wall of the tube such that the axis of the passage is parallel to, but radially offset from, an axis of the tube,
whereby when the inhaler is positioned with the passage below the tube and the axis of the passage is substantially vertical, a capsule located in the tube is guided by the ramp surface towards the inlet end of the passage.

81. A unit dosage form containing a pharmaceutical composition as described in claim 44.

82. The unit dosage form of claim 81, in the form of a capsule.

83. The dosage form of claim 82, wherein the capsule is a hydroxypropyl methylcellulose capsule.

84. A nasal drug delivery device, comprising: a pharmaceutical formulation as described in claim 60, and a means for housing and dispensing metered unit dosages of the formulation via a patient's nasal opening.

85. A nasal drug delivery device, comprising: a pharmaceutical formulation as described in claim 67, and a means for housing and dispensing metered unit dosages of the formulation into a patients nasal passages and/or sinuses.
